(19) United States
(12) Patent Application Publication
(10) Pub. No.: US 2024/0374278 A1
(43) Pub. Date: Nov. 14, 2024

(54) DEVICES AND METHODS FOR CUTTING A PERCUTANEOUS IMPLANT

(71) Applicant: THE REGENTS OF THE UNIVERSITY OF CALIFORNIA, Oakland, CA (US)

(72) Inventor: Roger E. Goldman, Oakland, CA (US)

(21) Appl. No.: 18/565,971

(22) PCT Filed: Jun. 23, 2022
(86) PCT No.: PCT/US22/34629

§ 371 Date: Nov. 30, 2023

Related U.S. Application Data
(60) Provisional application No. 63/214,686, filed on Jun. 24, 2021.

(51) Int. Cl. A61B 17/32; A61B 17/3211; A61J 15/00

(52) U.S. Cl. CPC A61B 17/320016; A61B 17/3211; A61J 15/0015; A61B 2017/320052; A61B 2017/32113

(57) ABSTRACT

Surgical devices and methods for cutting a percutaneous implant are disclosed. A surgical device may include a proximal end having a handle, and a distal end having a first arm. a second arm, and a cutting element. The cutting element may be disposed partially or entirely between the first arm and the second arm. At least of portion of the first arm and/or the second arm may be distal to the cutting element to prevent or inhibit the cutting element from cutting tissue adjacent to the percutaneous implant during use of the surgical device to cut the percutaneous implant. A method may include inserting at least a portion of a surgical device into an opening in an ex vivo portion of a percutaneous implant and using the surgical device to create at least one continuous cut through the ex vivo portion and an in vivo portion of the percutaneous implant.

### CROSS-REFERENCE TO RELATED APPLICATION

This application claims priority to U.S. Provisional Application No. 63/214,686, filed Jun. 24, 2021, the disclosure of which is herein incorporated by reference in its entirety for all purposes.

### FIELD OF DISCLOSURE

The present disclosure generally relates to surgical devices and methods pertaining to percutaneous implants, and, more particularly, the removal of such implants.

### BACKGROUND

A percutaneous implant is an object foreign to the body that is placed through the skin and other tissue to create a permanent or semi-permanent pathway or port through the skin into the body. An example of a percutaneous implant is a gastrostomy tube. There are various types of gastrostomy tubes including, for example, percutaneous endoscopic gastrostomy (PEG) tubes, radiologically inserted gastrostomy (RIG) tubes, and a surgically placed gastrostomy tubes. Generally a gastrostomy tube is a device placed through the abdominal wall into the stomach of a patient to allow food, fluid, drugs, and/or other substances to be introduced or administered directly into the stomach, or to allow ventilation and drainage in the case of a downstream bowel obstruction. Gastrostomy tubes are indicated when, for example, ingestion via the mouth is insufficient or impossible and the time to recovery of per oral feeding is expected to be too long for nasoenteric tubes.

A gastrostomy tube typically has an anchoring element that is positioned within the stomach to prevent inadvertent removal. The anchoring element may be comprised of an inflatable balloon, a reducible flange, a Cope loop, or a non-reducible radially extending flange. A non-reducible anchoring element, however, can present challenges when the time comes for proper removal. One conventional method of removing a gastrostomy tube, often referred to as the “cut and push” method, involves severing a portion of the gastrostomy tube outside of the body and pushing a remainder of the gastrostomy tube into the stomach, in the hopes that this portion of the gastrostomy tube, which includes the anchoring element, passes naturally through the patient's gastrointestinal (GI) tract. This method involves a risk that the gastrostomy tube does not pass naturally and creates an obstruction in and/or perforates the GI tract and/or has other undesirable effects. To mitigate such complications, in certain cases an endoscopic or surgical procedure will be performed to retrieve the retained portion of the gastrostomy tube. Such procedures are invasive and for frail patients may not be possible due to the risk of exacerbating other medical conditions. An alternative method for removal, traction removal, is associated with tearing along the stoma tract, pain, bleeding, and site infection.

Accordingly, there is a need for improved tools and methods for removing percutaneous implants such as gastrostomy tubes. The present disclosure sets forth devices and methods embodying advantageous alternatives to existing devices and methods for removing percutaneous implants that address one or more of the challenges or needs mentioned herein, as well as provide other benefits and advantages.

### SUMMARY

One aspect of the present disclosure provides a surgical device for cutting a percutaneous implant, including, but not limited to, a gastrostomy tube such as, for example, a percutaneous endoscopic gastrostomy (PEG) tube, a radiologically inserted gastrostomy (RIG) tube, and/or a surgically placed gastrostomy tube. The surgical device may include a proximal end and a distal end. The proximal end may include a handle. The distal end may include a first arm, a second arm, and a cutting element. The cutting element may be disposed partially or entirely between the first arm and the second arm. At least a portion of the first arm and/or at least a portion of the second arm may be distal to the cutting element to prevent or inhibit the cutting element from cutting or otherwise injuring tissue adjacent to the percutaneous implant during use of the surgical device to cut the percutaneous implant.

The first cutting element may include a first blade, and the first blade may extend partially or entirely between an inner surface of the first arm and an inner surface of the second arm. Furthermore, a position of the first cutting may be fixed with respect to the first arm and/or the second arm at least during use of the surgical device to cut the percutaneous implant.

A portion of or the entirety of the first arm may be spaced from a portion of or the entirety of the second arm by a first distance. The first distance may larger than a wall thickness of a portion of or the entirety of the percutaneous implant.

The surgical device may further include a second cutting element. At least a portion of the first cutting element may be disposed between the first arm and the second arm and/or between the first arm and a third arm, and at least a portion of the second cutting element may be disposed between the first arm and the second arm and/or between the second arm and the third arm. A position of the first cutting element and a position of the second cutting element may be fixed with respect to each other during use of the surgical device to cut the percutaneous implant.

The second cutting element may include a second blade, and the second blade may extend partially or entirely between an inner surface of the second arm and a surface of the third arm.

Another aspect of the present disclosure provides a method of cutting a percutaneous implant coupled to a patient, including, but not limited to, gastrostomy tube such as, for example, a percutaneous endoscopic gastrostomy (PEG) tube, a radiologically inserted gastrostomy (RIG) tube, and/or surgically placed gastrostomy tube coupled to a patient. The method may include inserting at least a portion of a surgical device into an opening in an ex vivo portion of a percutaneous implant. The method may further include using the surgical device to create at least one continuous cut through the ex vivo portion and an in vivo portion of the percutaneous implant. The method may include cutting through the percutaneous implant without cutting or otherwise injuring tissue adjacent to the percutaneous implant.

The method may additionally include withdrawing the surgical device from the percutaneous implant and subsequently pulling the in vivo portion of the percutaneous implant out of the patient or pushing the ex vivo portion of the percutaneous implant inside of the patient so that, for example, the percutaneous implant passes naturally through the patient's GI tract.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicative of the presence or absence of particular elements in any of the exemplary embodiments, except as may be explicitly delineated in the corresponding written description. Also, none of the drawings are necessarily to scale.

FIG. 1 is a side view of a percutaneous implant in a patient's body, the latter of which is depicted in cross-section.

FIG. 2 is a perspective view of a device for cutting a percutaneous implant according to an embodiment of the present disclosure.

FIG. 3 is another perspective view of the device in FIG. 2.

FIG. 4 is another perspective view of the device in FIG. 2, illustrating only a distal end of this device.

FIG. 5 is a side view of the distal end of the device in FIG. 2.

FIG. 6 is a perspective view of the device in FIG. 2 being used to cut a percutaneous implant.

FIG. 7 is another perspective view of what is shown in FIG. 6.

FIG. 8 is a perspective view of a device for cutting a percutaneous implant according to another embodiment of the present disclosure.

FIG. 9 is another perspective view of the device in FIG. 8, depicting only a distal end of this device.

FIG. 10 is a side view of the distal end of the device in FIG. 8.

FIG. 11 is a perspective view of a device for cutting a percutaneous implant according to another embodiment of the present disclosure.

FIG. 12 is an enlarged view of the distal end of the device shown in FIG. 11.

FIG. 13 is a top view of a distal end of the device illustrated in FIG. 11.

FIG. 14 is a rear view of a proximal end of the device depicted in FIG. 11.

FIG. 15 is a side view of the proximal end of the device shown in FIG. 11.

FIG. 16 is a top view of the proximal end of the device illustrated in FIG. 15.

FIG. 17 is a rear view of an alternative embodiment of the proximal end of the device depicted in FIG. 14.

FIG. 18 is a side view of the proximal end of the device illustrated in FIG. 17.

FIG. 19 is a perspective view of a device for cutting a percutaneous implant according to another embodiment of the present disclosure.

FIG. 20 is a side view the distal end of the device depicted in FIG. 19.

FIG. 21 is a top view of the distal end of the device shown in FIG. 19.

FIG. 22 is a front view of the distal end of the device illustrated in FIG. 19.

### DETAILED DESCRIPTION

The present disclosure generally relates to devices and methods for use in removing a percutaneous implant, including, for example, cutting a percutaneous implant to release or detach it from surrounding tissue. The presently disclosed devices and methods may allow a surgeon, surgical assistant, and/or other user to create one or more cuts (e.g., incisions, perforations, etc.) in a percutaneous implant, including a portion of the percutaneous implant located beneath the surface of a patient's skin, without having to approach the percutaneous implant from inside the body via, for example, an endoscopic or surgical procedure. Instead, the user may cut the percutaneous implant starting with a portion of the implant located outside of the body, followed by cutting a portion of the implant located inside of the body. Moreover, the devices and methods disclosed herein may allow one to cut a percutaneous implant with little or no risk of cutting or damage to tissue adjacent to (e.g., surrounding) the percutaneous implant. In the case of removing of a gastrostomy tube, the ability to cut the portion of the gastrostomy tube inside of the patient's body decreases or eliminates the risk that this portion of the tube, which may be mushroom-shaped prior to being cut, will obstruct and/or injure the gastrointestinal (GI) tract when later passing there through. For example, the cut portion of the tube may be more likely to bend, deform, and/or change shape in other ways to facilitate passage of the portion of the tube through the gastrostomy stoma or through the GI tract. Additional benefits, advantages, and uses of the presently disclosed devices and methods are possible and are not limited to those mentioned herein.

As used herein, the terms “generally,” “substantially,” “approximately,” “about,” and similar terms are intended to have a broad meaning in harmony with the common and accepted usage by those of ordinary skill in the art to which the subject matter of this disclosure pertains. It should be understood by those skilled in the art who review this disclosure that these terms are intended to allow a description of certain features described and claimed without restricting the scope of these features to precise numerical ranges. Accordingly, these terms should be interpreted as indicating that insubstantial or inconsequential modifications or alterations of the subject matter described and claimed are considered to be within the scope of the invention as recited in the appended claims.

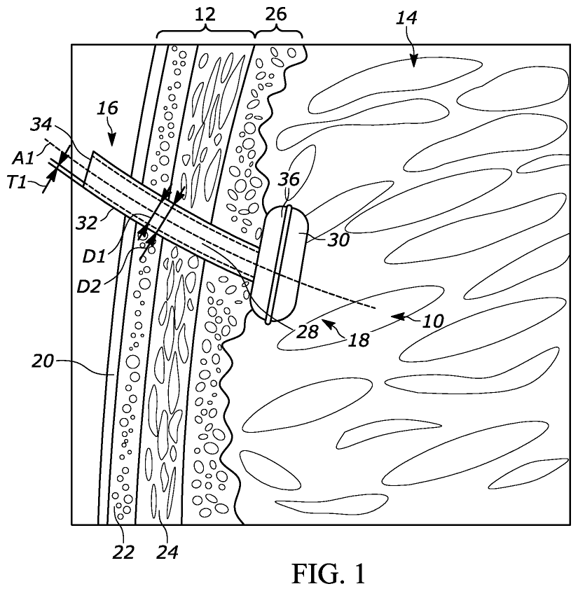

FIG. 1 illustrates an example of a percutaneous implant 10 for which the presently disclosed devices and methods are useful in removing. The percutaneous implant 10 depicted in FIG. 1 is a gastrostomy tube; however, the devices and methods disclosed herein are not limited to removing gastrostomy tubes. Being a gastrostomy tube, the percutaneous implant 10 may be placed through a patient's abdominal wall 12 into the stomach 14 and may be configured to allow food, fluid, drugs, and/or other substances to be introduced or administered directly into the stomach 14 or allow substances to egress from the stomach. The percutaneous implant 10 may be any type of gastrostomy tube, including, but not limited to, a percutaneous endoscopic gastrostomy (PEG) tube, a radiologically inserted gastrostomy (RIG) tube, and/or a surgically placed gastrostomy tube.

Generally the percutaneous implant 10 includes an ex vivo portion 16 and an in vivo portion 18. The ex vivo portion 16 includes any portion of the percutaneous implant 10 located outside of the patient's body; whereas, the in vivo portion 18 includes any portion of the percutaneous implant 10 located inside of the patient's body including any portion of the of the percutaneous implant 10 disposed through the abdominal wall 12 (including, but not limited to, the skin 20, the subcutaneous tissue 22, and the abdominal muscle 24) as well as any portion of the percutaneous implant 10 disposed through the stomach wall 24 and any portion of the percutaneous implant 10 disposed within an interior space of the stomach 14. As an example, the in vivo portion 18 may be disposed at least through a stoma formed in the abdominal wall 12 and/or stomach wall 24. In embodiments where the percutaneous implant 10 is a PEG tube, initial placement of the percutaneous implant 10 may be achieved, for example, through a gastroscopic procedure in which, for example, an endoscope is inserted through the esophagus into the stomach, then creates a stoma first through the stomach wall 24 and then through abdominal wall 12, and then inserts the percutaneous implant 10 into and through the stoma such that the ex vivo portion 16 of the percutaneous 10 is positioned outside of the patient's body. In embodiments where the percutaneous implant 10 is a RIG tube or a surgically placed gastrostomy tube, initial placement of the percutaneous implant 10 may be achieved by, for example, creating a stoma first through the abdominal wall 12 and then through the stomach wall 24, then inserting the percutaneous implant 10 through the stoma into the stomach 14, and thereafter deploying an anchor portion of the percutaneous implant 10 to secure the percutaneous implant 10 with respect to the stomach wall 24.

The percutaneous implant 10 may include at least: a tubular portion 28 configured to extend through the abdominal wall 12 and the stomach wall 14; and an anchor portion 30 configured to be disposed within the interior space of the stomach 14. The tubular portion 28 may include an annular wall 32 extending along and/or centered about a longitudinal axis A1 of the percutaneous implant 10. The annular wall 32 may have a thickness T1, which, as discussed below, may be less than a distance between two arms of the presently disclosed surgical device. As an example, the thickness T1 of the annular wall 32 may be: in a range between approximately (e.g., ±10% or ±5%) 0.1-3 mm, or in a range between approximately (e.g., ±10% or ±5%) 0.1-2 mm, or in a range between approximately (e.g., ±10% or ±5%) 0.1-1.5 mm, or in a range between approximately (e.g., ±10% or ±5%) 0.1-1 mm, or in a range between approximately (e.g., ±10% or ±5%) 0.1-0.5 mm, or in a range between approximately (e.g., ±10% or ±5%) 0.1-0.3 mm, or less than or equal to approximately (e.g., ±10% or ±5%) 3 mm, or less than or equal to approximately (e.g., ±10% or ±5%) 2 mm, or less than or equal to approximately (e.g., ±10% or ±5%) 1 mm, or less than or equal to approximately (e.g., ±10% or ±5%) 0.5 mm, or any other suitable dimension.

A central passage or opening 34 may extend through the tubular portion 28 and the anchor portion 30 to provide a direct conduit between the outside of the body and the inside of the stomach 14. As an example, the opening 34 may extend along and/or be centered about the longitudinal axis A1 of the percutaneous implant 10. As a more specific example, an inlet of the percutaneous implant 10 may be defined at least in part by the portion of the opening 34 extending through the tubular portion 28, and an outlet of the percutaneous implant 10 may be defined at least in part by the portion of the opening 34 extending through anchor portion 30. The opening 34 may have an inner diameter D1. The annular wall 32 of the tubular portion 28 may have an outer diameter D2. As an example, the inner diameter D1 and/or the outer diameter D2 may be: in a range between approximately (e.g., ±10% or ±5%) 1-12 mm, or in a range between approximately (e.g., ±10% or ±5%) 2-10 mm, or in a range between approximately (e.g., ±10% or ±5%) 3-9 mm, or in a range between approximately (e.g., ±10% or ±5%) 4-8 mm, or in a range between approximately (e.g., ±10% or ±5%) 4-6 mm, or in a range between approximately (e.g., ±10% or ±5%) 5-6 mm, or less than or equal to approximately (e.g., ±10% or ±5%) 12 mm, or less than or equal to approximately (e.g., ±10% or ±5%) 10 mm, or less than or equal to approximately (e.g., ±10% or ±5%) 9 mm, or less than or equal to approximately (e.g., ±10% or ±5%) 8 mm, or less than or equal to approximately (e.g., ±10% or ±5%) 7 mm, or less than or equal to approximately (e.g., ±10% or ±5%) 6 mm, or less than or equal to approximately (e.g., ±10% or ±5%) 5 mm, or less than or equal to approximately (e.g., ±10% or ±5%) 4 mm, or less than or equal to approximately (e.g., ±10% or ±5%) 3 mm, or any other suitable dimension. In some embodiments, the opening 34 in tubular portion 28 may be selectively covered with a cap or other closure member or cover member (not shown in FIG. 1), so as to seal close the opening 34 in tubular portion 28 when the percutaneous implant 10 is not being used to introduce food, liquid(s), drug(s), etc. into the stomach 14, or allow egress of stomach contents.

The anchor portion 30 may be configured to prevent or inhibit the in vivo portion 18 from being pulled out of the patient's body. As an example, the anchor portion 30 may have an outer diameter or other dimension transverse to the longitudinal axis A1 that is larger than a corresponding dimension of the tubular portion 30. As a more specific example, the anchor portion 30 may include a flange 36 extending radially outwardly from an axial end of the tubular portion 28 and which may contact an inner surface of the stomach wall 26, as seen in FIG. 1. As an even more specific example, when viewed from the side, the anchor portion 30 may be generally mushroom-shaped or generally shaped like a mushroom head. Depending on the shape of the anchor portion 30, the percutaneous implant 10 may be referred to as a “mushroom-retained gastrostomy tube” in some contexts. The anchor portion 30 may be relatively rigid, due to, for example, its material and/or geometry, such that the anchor portion 30 resists deformation and/or being pulled through the stoma in the abdominal wall 12 and/or stomach wall 26 if the tubular portion 28 is pulled in a direction away from the patient's body.

The percutaneous implant 10 may be made partially or entirely of silicone, polyurethane, or any other suitable polymer or material, or any combination thereof.

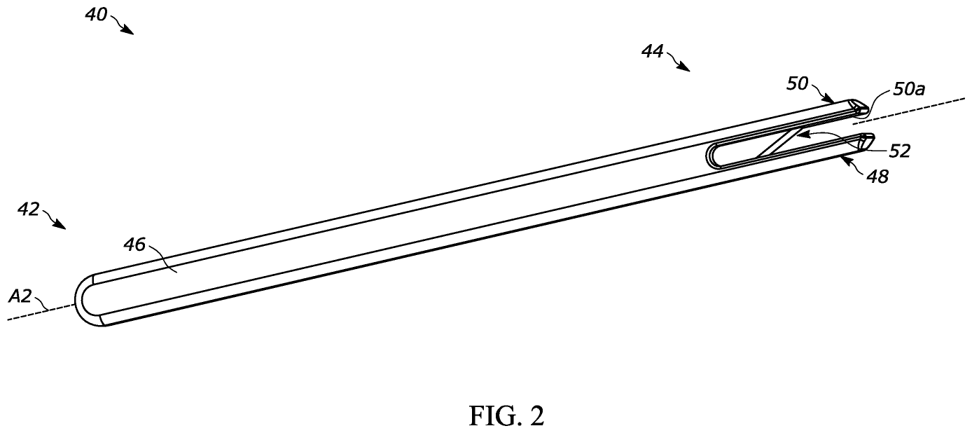

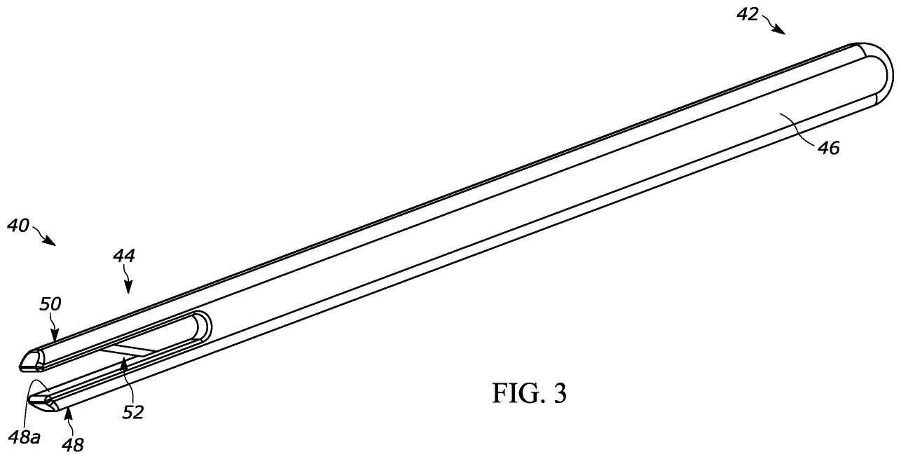

Turning to FIGS. 2-10, various embodiments of a surgical device and method for removing a percutaneous implant, including, but not limited to, the percutaneous implant 10 described above, will now be described. Any of the embodiments of the surgical device described herein may be included (e.g., packaged) with a percutaneous implant, including, for example, the percutaneous implant 10, as part of a kit. As used herein, the term “surgical device” encompasses a surgical tool, a surgical instrument, a surgical apparatus, and the like.

FIGS. 2-5 illustrate a first embodiment of a surgical device 40 in accordance with principles of the present disclosure. The surgical device 40 generally is configured to create one or more cuts (e.g., incisions, perforations, etc.) in a percutaneous implant, and may have other uses and functions as well, including, for example, cutting other types of implants, tubing, and/or items which are not implanted in a patient. The surgical device 40 may include a longitudinal axis A2 and a proximal end 42 and a distal end 44 arranged at respective positions along the longitudinal axis A2. The longitudinal axis A2 of the present embodiment may be linear; however, in other embodiments, the longitudinal axis A2 may be partially or entirely non-linear, including, for example, being curved or curvilinear. The surgical device 40 may have a generally elongate shape, such that, for example, an overall or maximum length of the surgical device 40 in a direction along longitudinal axis A2 is significantly larger than an overall or maximum width of the surgical device in a direction transverse (e.g., perpendicular) to the longitudinal axis A2.

The proximal end 42 of the surgical device 40 may include a handle 46 configured to allow a user (e.g., a surgeon, a surgical assistant, a doctor, a nurse practitioner, a nurse, etc.) to maneuver and/or manipulate the surgical device 40 during use of the surgical device 40 to cut a percutaneous implant. As an example, the handle 46 may be ergonomically sized, shaped, textured, and/or dimensioned to be held and/or gripped by one or more or hands of a person including, for example, one or more fingers of the person. Additionally or alternatively, the handle 46 may configured to be coupled to an external device or machine. The handle 46 may be constructed of a rigid material including, for example, metal (e.g., stainless steel and/or surgical steel), certain plastics (e.g., polycarbonate), ceramic, or any other suitable material, or any combination thereof.

The distal end 44 of the surgical device 40 may include a first arm 48, a second arm 50, and a cutting element 52. The first arm 48 and/or the second arm 50 generally may extend longitudinally along the longitudinal axis A2 of the surgical device 40. As an example, a portion of or the entirety of the first arm 48 and/or a portion of or the entirety of the second arm 50 may be parallel or substantially parallel to the longitudinal axis A2 of the surgical device 40. Additionally or alternatively, a portion of or the entirety of the first arm 48 may be parallel or substantially parallel to a portion of or the entirety of the second arm 50. The first arm 48 may be spaced from the second arm 50 by a distance Z. As an example, an inner surface 48a of the first arm 50 may be spaced from an inner surface 50a of the second arm 50 by the distance Z. The distance Z may be constant traveling in a direction along the longitudinal axis A2, or, alternatively, may vary traveling in a direction along the longitudinal axis A2.

The distance Z between the first arm 48 and the second arm 50 may be selected such that at least a portion of the percutaneous implant 10 can fit between the first arm 48 and the second arm 50 during use of the surgical device 40 to cut the percutaneous implant 10. As an example, the distance Z between the first arm 48 and the second arm 50 may be larger than: the thickness T of the annular wall 32 of the tubular portion 28 of the percutaneous implant 10 and/or the outer diameter D2 of the tubular portion 28 of the percutaneous implant 10. As an even more specific example, the distance Z between the first arm 48 and the second arm 50 may be: in a range between approximately (e.g., ±10% or ±5%) 1-25 mm, or in a range between approximately (e.g., ±10% or ±5%) 1-20 mm, or in a range between approximately (e.g., ±10% or ±5%) 1-15 mm, or in a range between approximately (e.g., ±10% or ±5%) 1-10 mm, or in a range between approximately (e.g., ±10% or ±5%) 5-25 mm, or in a range between approximately (e.g., ±10% or ±5%) 5-20 mm, or in a range between approximately (e.g., ±10% or ±5%) 5-15 mm, or larger than approximately (e.g., ±10% or ±5%) 2 mm, or larger than approximately (e.g., ±10% or ±5%) 5 mm, or larger than approximately (e.g., ±10% or ±5%) 7.5 mm, or larger than approximately (e.g., ±10% or ±5%) 10 mm, or larger than approximately (e.g., ±10% or ±5%) 12 mm, or larger than approximately (e.g., ±10% or ±5%) 15 mm, or larger than approximately (e.g., ±10% or ±5%) 20 mm, or any other suitable dimension.

The first arm 48 may and/or the second arm 50 may have a cross-sectional shape, taken in an imaginary plane perpendicular to the longitudinal axis A2, that is rectangular, square, hemi-spherical, circular, or any other suitable shape. In the present embodiment, this cross-sectional shape is generally rectangular for each of the first arm 48 and the second arm 50. As such, the first arm 48 has a width W1 and a height H1, and the second arm 50 has a width W2 and a height H2. In alternative embodiments, for example, where the cross-sectional shape of the first arm 48 and/or the second arm 50 is circular, the width W1 and/or height H1 may correspond to a diameter of the first arm 48 and the width W2 and/or height H2 may correspond to a diameter of the second arm 50.

The first arm 48 and/or the second arm 50 may be dimensioned so that it can be inserted, partially or entirely, into and/or through the opening 34 in the percutaneous implant 10. As an example, any one or any combination of the width W1, the height H1, the width W2, and the height H2 may be less than the inner diameter D1 of the opening 34 in the tubular portion 28 and/or the anchor portion 30 of the percutaneous implant 10. As a more specific example, any one or any combination of the width W1, the height H1, the width W2, and the height H2 may be: in a range between approximately (e.g., ±10% or ±5%) 1-12 mm, or in a range between approximately (e.g., ±10% or ±5%) 2-10 mm, or in a range between approximately (e.g., ±10% or ±5%) 3-9 mm, or in a range between approximately (e.g., ±10% or ±5%) 4-8 mm, or in a range between approximately (e.g., ±10% or ±5%) 4-6 mm, or in a range between approximately (e.g., ±10% or ±5%) 5-6 mm, or less than or equal to approximately (e.g., ±10% or ±5%) 12 mm, or less than or equal to approximately (e.g., ±10% or ±5%) 10 mm, or less than or equal to approximately (e.g., ±10% or ±5%) 9 mm, or less than or equal to approximately (e.g., ±10% or ±5%) 8 mm, or less than or equal to approximately (e.g., ±10% or ±5%) 7 mm, or less than or equal to approximately (e.g., ±10% or ±5%) 6 mm, or less than or equal to approximately (e.g., ±10% or ±5%) 5 mm, or less than or equal to approximately (e.g., ±10% or ±5%) 4 mm, or less than or equal to approximately (e.g., ±10% or ±5%) 3 mm, or any other suitable dimension.

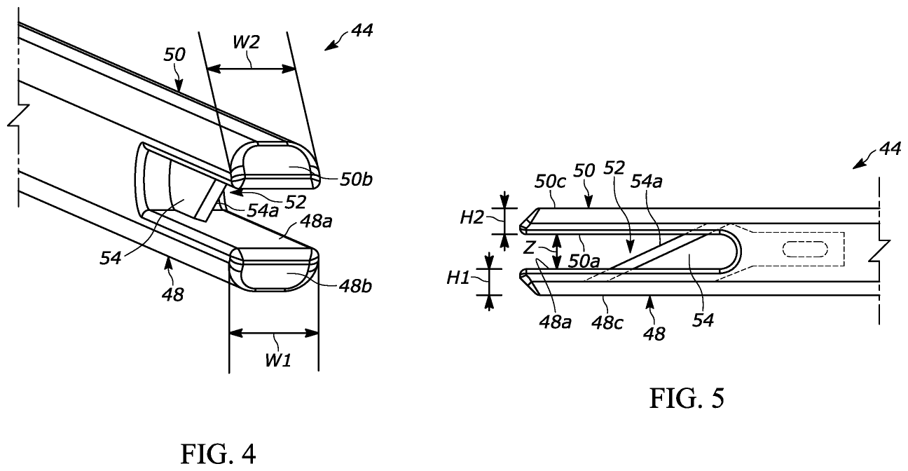

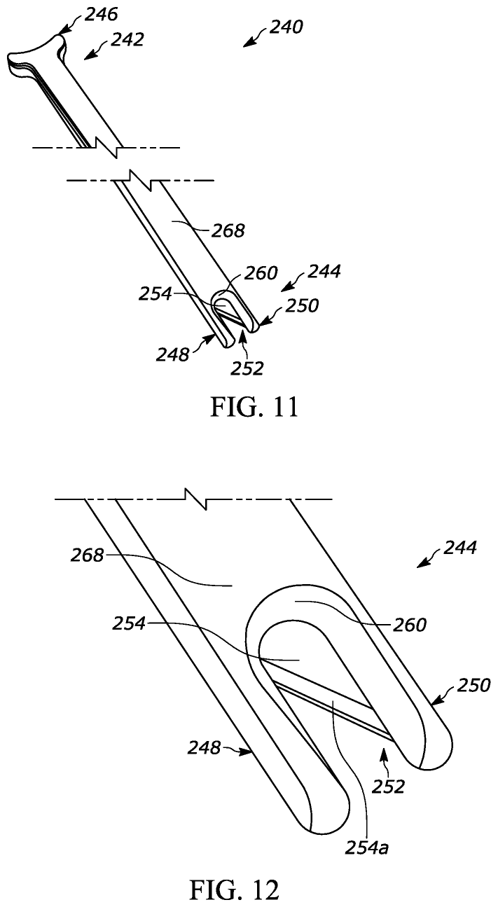

In the embodiment illustrated in FIG. 4, the width W1 of the first arm 48 equals or is substantially the same as the width W2 of the second arm 50. In other embodiments, including, for example, the embodiment illustrated in FIG. 11, the width W1 of the first arm 48 or a portion thereof may differ from the width W2 of the second arm 50 or a portion thereof. As an example, the width W1 of the first arm 48 may be less than the width W2 of the second arm 50, or vice versa. During use, the one of the arms 48 and 50 having the smaller width may be inserted into the tubular portion 28 of the percutaneous implant 10, whereas the other one of the arms 48 and 50 may slide along or be positioned adjacent to an exterior surface of the tubular portion 28 of the percutaneous implant 10, providing lateral stability due to its larger width. In some embodiments, the width W1 of the first arm 48 may be less than or equal to an inner diameter of the tubular portion 28 of the percutaneous implant 10, whereas the width W2 of the second arm 50 may be equal to or larger than an outer diameter of the tubular portion 28 of the percutaneous implant 10.

The first arm 48 may include a distally facing end surface 48b, and the second arm 50 may include a distally facing end surface 50b. As an example, the distally facing end surface 48b and/or the distally facing end surface 50b may define a distal-most surface of the surgical device 40. The distally facing end surface 48b and/or the distally facing end surface 50b may be configured to facilitate insertion into the opening 34 of the percutaneous implant 10 and/or between an outer surface of the percutaneous implant 10 and tissue immediately adjacent to the outer surface of the percutaneous implant 10, including, for example, without cutting or otherwise causing injury to the tissue immediately adjacent to the percutaneous implant 10. As an example, the distally facing end surface 48b and/or the distally facing end surface 50b may be partially or entirely arranged at a non-perpendicular angle with respect to the longitudinal axis A2, as seen in FIG. 4, so that the distally facing end surface 48b and/or the distally facing end surface 50b is able to gradually push tissue away from the outer surface of the percutaneous implant 10 to make space for the first arm 48 and/or the second arm 50 during use of the surgical device 40 to cut the percutaneous implant 10. Additionally or alternatively, one or more edges of the distally facing end surface 48b and/or the distally facing end surface 50b may possess a rounded geometry to, for example, reduce the risk of injury to the tissue immediately adjacent to the percutaneous implant 10.

Generally, the cutting element 52 is configured to create one or more cuts in the percutaneous implant 10 during insertion of the first arm 48 and/or the second arm 50 into and/or through the patient's body and/or the opening 34 in the percutaneous implant 10. As an example, a portion of or the entirety of the cutting element 52 may be disposed between the first arm 48 and the second arm 50, such that the cutting element 52 spans the entirety of or a portion of the distance Z between the first arm 48 and the second arm 50. As a more specific example, the cutting element 52 may extend at least between the inner surface 48a of the first arm 48 and the inner surface 50a of the second arm 50. The cutting element 52 may be coupled (e.g., removably coupled or permanently coupled) to the first arm 48, the second arm 50, and/or another portion of the surgical device 40. As an example, the cutting element 52 may be coupled to the first arm 48 and/or the second arm 50 such that a position of the cutting element 52 is fixed (e.g., stationary or immovable) with respect to a position of the first arm 48 and/or a position of the second arm 50 at least during use of the surgical device 40 to cut the percutaneous implant 10. In some embodiments, no portion of the surgical device 40 may move with respect to another portion of the surgical deice 40 during use of the surgical device 40 to cut the percutaneous implant 10. Alternative embodiments of the surgical device 40, however, may include one or more moving parts.

A distal end of the cutting element 52 may include a sharpened edge or other pointed geometry allowing the distal end of the cutting element 52 to form one or more cuts in the percutaneous implant 10. As an example, the distal end of the cutting element 52 may be configured to form one or cuts in the annular wall 32 of the percutaneous implant 10 when the annular wall 32 is positioned between the first arm 48 and the second arm 50 and/or when the surgical device 40 is moved in a distal direction along the longitudinal axis A1 of the percutaneous implant 10. As a more specific example, the distal end of the cutting element 52 (or the entirety of the cutting element 52) may comprise a blade 54. As a more specific example, the distal end of the blade 54 may be sharpened and/or a distally facing end surface 54a of the blade 54 (in whole or in part) may be arranged at a non-perpendicular angle with respect to the longitudinal axis A2 of the surgical device 40. This angle may be, for example: in a range between approximately (e.g., ±10% or ±5%) 15-75 degrees, or in a range between approximately (e.g., ±10% or ±5%) 30-75 degrees, or in a range between approximately (e.g., ±10% or ±5%) 30-60 degrees, or in a range between approximately (e.g., ±10% or ±5%) 40-50 degrees, or less than or equal to approximately (e.g., ±10% or ±5%) 75 degrees, or less than or equal to approximately (e.g., ±10% or ±5%) 60 degrees, or less than or equal to approximately (e.g., ±10% or ±5%) 50 degrees, or less than or equal to approximately (e.g., ±10% or ±5%) 45 degrees, or any other suitable angle. The distally facing end surface 54a of the blade 54 may be configured to create (e.g., form) at least one continuous (e.g., uninterrupted) cut through any one or combination of: the ex vivo portion 16 of the percutaneous implant 10, the in vivo portion 18 of the percutaneous implant 10, the tubular portion 28 of the percutaneous implant 10, and the anchor portion 30 of the percutaneous implant 10. Furthermore, the blade 54 may be constructed of any suitably rigid and/or hard material including, for example, metal (e.g., stainless steel and/or surgical steel), certain plastics (e.g., polycarbonate), ceramic, or any other suitable material, or any combination thereof.

The first arm 48 and/or the second arm 50 may protect or guard tissue (e.g., the skin 20, subcutaneous tissue 22, abdominal muscle 24, stomach wall 26) surrounding or otherwise adjacent to the percutaneous implant 10 from being cut by the cutting element 52 while the cutting element 52 is being used to cut the percutaneous implant 10. This aspect of the first arm 48 and/or second arm 50 may be facilitated by, for example, the distal end of the first arm 48 and/or the distal end of the second arm 50 being distal to the distal end of the cutting element 52. As a more specific example, the distally facing end surface 48b of the first arm 48 and/or the distally facing end surface 50b of the second arm 50 may be distal to the distally facing end surface 54a of the blade 54, as seen in FIG. 5. As a result, the distally facing end surface 48b of the first arm 48 and/or the distally facing end surface 50b of the second arm 50 may be the leading penetration element(s) of the surgical device 40 as the surgical device 40 moves in the distal direction along the longitudinal axis A1 of the percutaneous implant 10 into the patient's body. This aspect of the first arm 48 and/or second arm 50, alone or in combination with the fact that the cutting element 52 is located between the first arm 48 and the second arm 50, enables the first arm 48 and/or the second arm 50 to push the patient's tissue outwardly (e.g., radially outwardly with respect to the longitudinal axis A1) away from the percutaneous implant 10 such that the cutting element 52 is inhibited or prevented from coming into contact with and/or cutting the tissue adjacent to the percutaneous implant 10 during use of the surgical device 40 to cut the percutaneous implant 10.

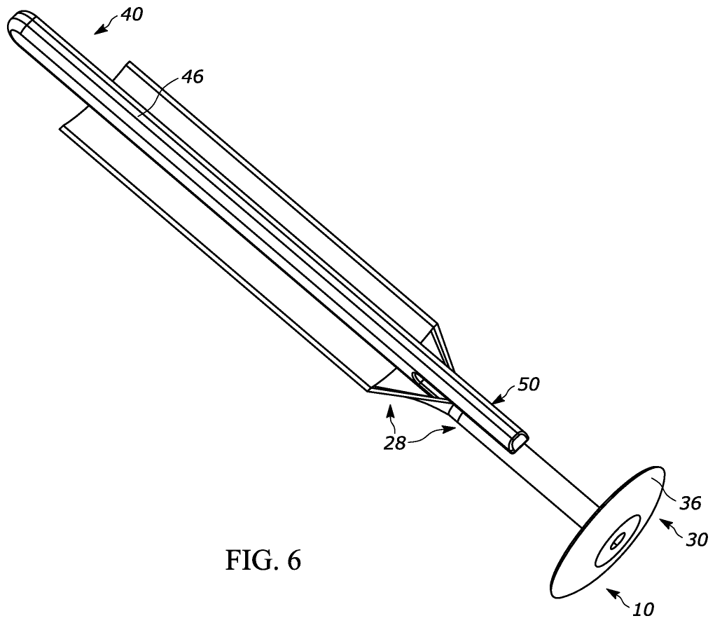

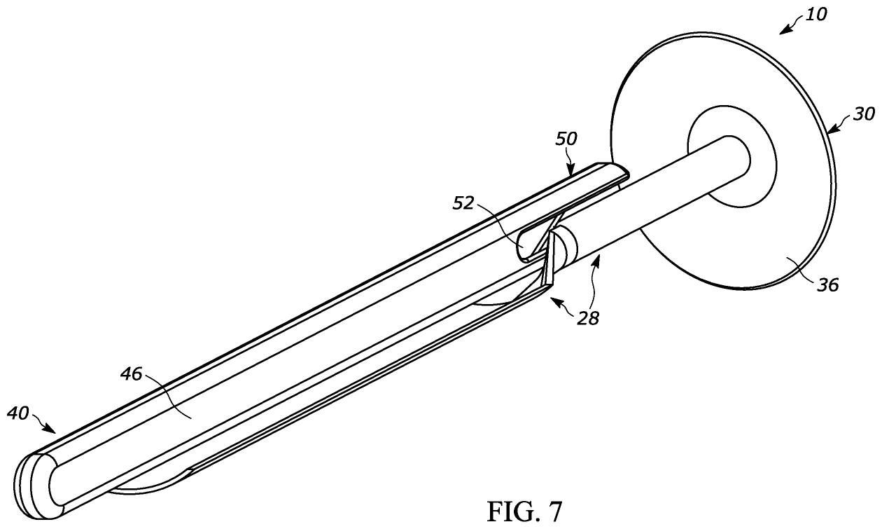

A method of using the surgical device 40 to cut a percutaneous implant will now be described with reference to FIGS. 6 and 7. The percutaneous implant depicted in FIGS. 6 and 7 has the same general features as the percutaneous implant 10 illustrated in FIG. 1, albeit with different dimensioning. Features illustrated in FIGS. 6 and 7 which are functionally and/or structurally similar to features depicted in FIG. 1 are identified with the same references as used in FIG. 1.

As an initial step, the surgical device 40 may be cleaned or sterilized so as to reduce or eliminate the possibility of the surgical device 40 introducing contaminants into the patient's body.

Next, a user (e.g., a surgeon, a surgical assistant, a doctor, a nurse practitioner, a nurse, etc.) may hold (e.g., grip) the handle 46 in his or her hand and optionally hold the tubular portion 28 of the percutaneous implant 10 in the other hand. Then, while continuing to hold the handle 46, the user may insert the distal end of the first arm 48 into the opening 34 in the tubular portion 28 of the percutaneous implant 10, or, if the percutaneous implant 10 does not include a tubular portion 28, into another opening in the ex vivo 16 of the percutaneous implant 10. Simultaneously or substantially simultaneously, or shortly thereafter, the distal end of the second arm 50 may be inserted in between patient's skin and the outer surface of the tubular portion 28 of the percutaneous implant 10. The step may, in some embodiments, involve stretching the skin and/or other tissue slightly to accommodate the second arm 50 but preferably does not involve cutting or otherwise injuring the skin and/or other tissue.

Subsequently, with the first arm 48 inserted into the opening 34, the user may push the surgical device 40 in the distal direction (i.e., toward the patient) generally along the longitudinal axis A1 of the percutaneous implant 10. Eventually this motion may cause the distally facing end surface 54a of blade 54 to come into contact with and begin cutting the annular wall 32 of the tubular portion 28 of the percutaneous implant 10. The user may continue moving the surgical device 40 in the distal direction, thereby causing the blade 54 to create a cut in the annular wall 32 or other ex vivo portion 16 of the percutaneous implant 10 that may be parallel or substantially parallel to the longitudinal axis A2 of the percutaneous implant 10. FIGS. 6 and 7 illustrate different views of a single moment in time after the surgical device 40 has cut through a proximal portion of the annular wall 32 of the percutaneous implant 10. The user may continue moving the surgical device 40 in the distal direction so that it continues to cut through the annular wall 32, including, eventually, cutting through the in vivo portion 18 of the percutaneous implant 10. In at least some embodiments, the blade 54 may not come into contact with the patient's skin or other tissue as it cuts through the in vivo portion 18 of the percutaneous implant 10 due to, for example, the second arm 50 having pushed the patient's skin and other tissue outwardly (e.g., radially outwardly with respect to the longitudinal axis A1).

As the surgical device 40 continues to move in the distal direction, eventually the second arm 50 may contact and push on the flange 36 of the anchor portion 30 of the percutaneous implant 10. This may cause at least a portion of the flange 36 to deform or collapse radially inwardly, such that the at least a portion of the flange 36 comes to be positioned between the first arm 48 and the second arm 50. Subsequently, the blade 54 may come into contact with the flange 36 and cut through the flange 36 and/or other portion(s) of the anchor portion 30. The user may continue to push the surgical device 40 in the distal direction until the blade 54 has cut through what was previously the radially outer edge of the flange 36 (or other portion) of the anchor portion 30 of the percutaneous implant 10. As a result, the surgical device 50 will have created, in a single distal motion or pass, a single continuous cut extending through the ex vivo portion 16 of the percutaneous implant 10 and the in vivo portion 18 of the percutaneous implant 10. As an example, this single continuous cut may extend uninterrupted from (and through) an end surface (e.g., an axial end surface) of the ex vivo portion 16 of the percutaneous implant 10 to (and through) an end surface of the in vivo portion 18 of the percutaneous implant 10.

After the cut has been created, the user may pull in the surgical device 40 in the proximal direction to remove the surgical device 40 from the percutaneous implant 10 and the patient's body. Optionally, the user then may repeat some or all of the foregoing steps to create a second, third, fourth, or more cuts in the percutaneous implant 10.

Next, after the surgical device 40 has been removed from patient's body, the user may pull the percutaneous implant 10 in the proximal direction to remove the in vivo portion 18 portion of the percutaneous implant 10 from the patient's body. This may be possible because the cut(s) created in the anchor portion 30 may allow the anchor portion 30 to deform and/or assume a shape facilitating passage of the anchor portion 30 through the stoma formed in the abdominal wall 12 and the stomach wall 26, with little or no attendant risk of injury to, for example, the abdominal wall 12 and/or the stomach wall 26. Alternatively, instead of pulling the percutaneous implant 10 out of the patient's body once cutting is complete, the user may push the ex vivo portion 16 into the patient's stomach 14, with the intent that the percutaneous implant 10 passes naturally through the patient's GI tract. This may be feasible because the one or more cuts created in the percutaneous implant 10 (including the tubular portion 28 and/or anchor portion 30) may allow the percutaneous implant 10 to deform and/or assume a shape facilitating passage of the per through the GI tract, without little or no attendant risk of, for example, the anchor portion 30 becoming immobilized in the GI tract due to its relatively large cross-sectional shape in its pre-cut form.

Lastly, after the percutaneous implant 10 has been removed, the user may suture close the stoma, insert a new percutaneous implant in the stoma, or allow the stoma site to heal by secondary intention.

In alternative embodiments, instead of inserting the first arm 48 into the opening 34 of the percutaneous implant 10, the second arm 48 may be inserted into the opening 34 and the first arm 48 may be positioned between the outer surface of the percutaneous implant 10 and the patient's tissue. The other steps of the above-described method may remain the same or substantially the same in such alternative embodiments.

In further alternative embodiments, neither the first arm 48 nor the second arm 50 may be inserted into the opening 34 of the percutaneous implant 10. Instead, the tubular portion 28 of the percutaneous implant 10 may be positioned between first arm 48 and the second arm 50. The other steps of the above-described method may remain the same or substantially the same in such alternative embodiments.

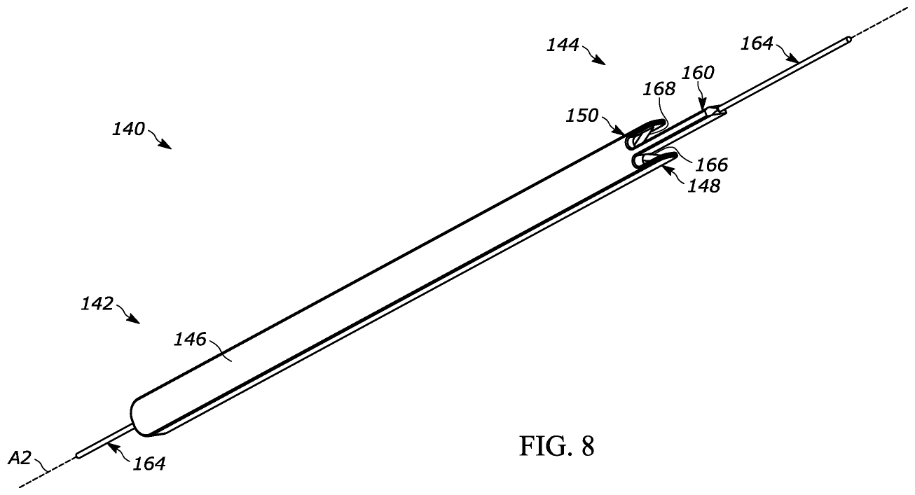

Referring now to FIGS. 8-10, a second embodiment of a surgical device, denoted with reference numeral 140, will now be described. Various elements of the surgical device 140 illustrated in FIGS. 8-10 may be similar or identical in structure, configuration, and/or function to elements of the surgical device 40 described above in conjunction with FIGS. 2-7. Such elements are assigned with the same reference numeral as used in FIGS. 2-7, except incremented by 100. A description of some of these elements is abbreviated or eliminated in the interest of conciseness. Details of the structure, configuration, and/or function that differentiate the embodiments of the surgical device 140 illustrated in FIGS. 8-10 from the embodiment of the surgical device 40 in FIGS. 2-7 are the focus of the discussion below.

The distal end 144 of the surgical device 140 may include a third arm 160 disposed at least partially between the first arm 148 and the second arm 150. As an example, at least a proximal end of the third arm 160 may be disposed between the first arm 148 and the second arm 150. As a more specific example, a proximal portion of a first surface 160a of the third arm 160 may face the inner surface 148a of the first arm 148 and a proximal portion of a second surface 160b of the third arm 160 may face the inner surface 150a of the second arm 150.

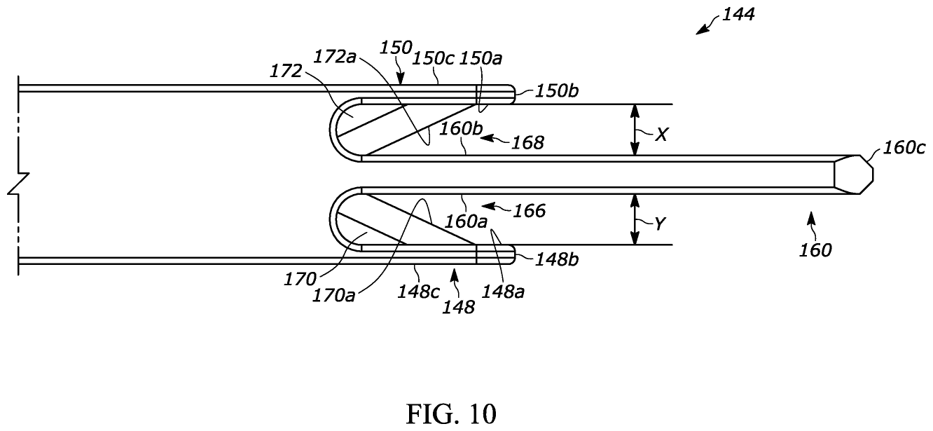

As seen in FIG. 10, the first surface 160a of the third arm 160 may be spaced from the inner surface 148a of the first arm 148 by a distance Y; and the second surface 160b of the third arm 160 may be spaced from the inner surface 150a of the second arm 150 by a distance X. The distance X and/or the distance Y may be selected such that at least a portion of the percutaneous implant 10 can fit between the first arm 148 and the third arm 160 and/or between the second arm 150 and the third arm 160 during use of the surgical device 140 to cut the percutaneous implant 10. As an example, the distance X and/or the distance Y may be larger than: the thickness T of the annular wall 32 of the tubular portion 28 of the percutaneous implant 10 and/or the outer diameter D2 of the tubular portion 28 of the percutaneous implant 10. As an even more specific example, the distance X and/or the distance Y may be: in a range between approximately (e.g., ±10% or ±5%) 1-25 mm, or in a range between approximately (e.g., ±10% or ±5%) 1-20 mm, or in a range between approximately (e.g., ±10% or ±5%) 1-15 mm, or in a range between approximately (e.g., ±10% or ±5%) 1-10 mm, or in a range between approximately (e.g., ±10% or ±5%) 5-25 mm, or in a range between approximately (e.g., ±10% or ±5%) 5-20 mm, or in a range between approximately (e.g., ±10% or ±5%) 5-15 mm, or larger than approximately (e.g., ±10% or ±5%) 2 mm, or larger than approximately (e.g., ±10% or ±5%) 5 mm, or larger than approximately (e.g., ±10% or ±5%) 7.5 mm, or larger than approximately (e.g., ±10% or ±5%) 10 mm, or larger than approximately (e.g., ±10% or ±5%) 12 mm, or larger than approximately (e.g., ±10% or ±5%) 15 mm, or larger than approximately (e.g., ±10% or ±5%) 20 mm, or any other suitable dimension.

Furthermore, the third arm 160 may be configured such that, during use of the surgical device 140 to cut a percutaneous implant, including, for example, the percutaneous implant 10, the third arm 160 is inserted into and/or comes into contact with the percutaneous implant 10 before any other portion of the surgical device 140. This may be achieved, for example, by making the third arm 160 longer (in the direction of the longitudinal axis A2) than the first arm 148 and/or the second arm 150. As an example, a distal end of the third arm 160 may be distal to a distal end of the first arm 148 and/or a distal end of the second arm 150. In some embodiments, the third arm 160 may have a width and/or height that is similar to or the same as the dimensions discussed above with respect to the first arm 48 and/or the second arm 50 of the surgical device 40.

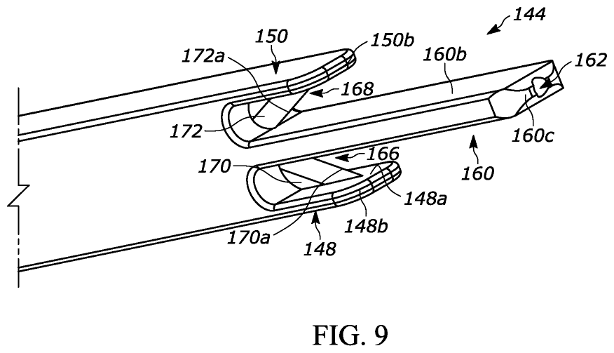

An opening 162 may be formed in a distally facing end surface 160c of the third arm 160, as seen in FIG. 9. The opening 162 may extend along and/or parallel to the longitudinal axis A2 between the distally facing end surface 160c and a proximal end surface of the proximal end 142 of the surgical device 140. As seen in FIG. 8, the opening 162 may permit a guidewire 164 to pass through the surgical device 140. As an example, the guidewire 164 may be inserted into patient's stomach 14 via the opening 34 in the percutaneous implant 10 and subsequently the surgical device 140 may be passed over the guidewire 164 such that the third arm 160 initially enters the opening 34 of the percutaneous implant 10 and subsequently the surgical device 140 cuts the percutaneous implant 10. Use of the guidewire 164 in this manner may provide the surgical device 140 with, for example, lateral stability during cutting. After cutting is complete and the percutaneous implant 10 is removed, the guidewire 164 may remain in place within the patient and thereby facilitate insertion of a new percutaneous implant into the patient, if desired.

As an aside, an opening similar to the opening 162 may be formed in the first arm 48 and/or the second arm 50 of the surgical device 40 described above and may be used with a guidewire in a similar manner as described here in connection with the surgical device 140.

The surgical device 140 may be configured to create two separate cuts, simultaneously or substantially simultaneous, in the percutaneous implant 10. As an example, the surgical device 140 may include a first cutting element 166 and a second cutting element 168, as seen in FIGS. 8-10. As an example, a portion of or the entirety of the first cutting element 166 may be disposed between the first arm 148 and the third arm 160, such that the first cutting element 166 spans the entirety of or a portion of the distance Y between the first arm 148 and the third arm 160. As a more specific example, the first cutting element 166 may extend at least between the inner surface 148a of the first arm 148 and the first surface 160a of the third arm 160. The first cutting element 166 may be coupled (e.g., removably coupled or permanently coupled) to the first arm 148, the third arm 160, and/or another portion of the surgical device 140. As an example, the first cutting element 166 may be coupled to the first arm 148 and/or the third arm 160 such that a position of the first cutting element 166 is fixed (e.g., stationary or immovable) with respect to a position of the first arm 148 and/or a position of the third arm 160 at least during use of the surgical device 140 to cut the percutaneous implant 10. Moreover, as an example, a portion of or the entirety of the second cutting element 168 may be disposed between the second arm 150 and the third arm 160, such that the second cutting element 168 spans the entirety of or a portion of the distance X between the second arm 150 and the third arm 160. As a more specific example, the second cutting element 168 may extend at least between the inner surface 150a of the second arm 150 and the second surface 160b of the third arm 160. The second cutting element 168 may be coupled (e.g., removably coupled or permanently coupled) to the second arm 150, the third arm 160, and/or another portion of the surgical device 140. As an example, the second cutting element 168 may be coupled to the second arm 150 and/or the third arm 160 such that a position of the second cutting element 168 is fixed (e.g., stationary or immovable) with respect to a position of the second arm 150 and/or a position of the third arm 160 at least during use of the surgical device 140 to cut the percutaneous implant 10.

A distal end of the first cutting element 166 and a distal end of the second cutting element 168 each may include a sharpened edge or other pointed geometry allowing the distal end of the first cutting element 166 and the distal end of the second cutting element 168 to form respective cuts in the percutaneous implant 10.

As an example, the distal end of the first cutting element 166 may be configured to form a first cut in the annular wall 32 of the percutaneous implant 10 when a first portion of the annular wall 32 is positioned between the first arm 148 and the third arm 160 and/or when the surgical device 140 is moved in a distal direction along the longitudinal axis A1 of the percutaneous implant 10. Similarly, the distal end of the second cutting element 168 may be configured to form a second cut in the annular wall 32 of the percutaneous implant 10 when a second portion of the annular wall 32 is positioned between the second arm 150 and the third arm 160 and/or when the surgical device 140 is moved in a distal direction along the longitudinal axis A1 of the percutaneous implant 10.

As a more specific example, the distal end of the first cutting element 166 (or the entirety of the first cutting element 166) may comprise a first blade 170 and/or the distal end of the second cutting element 168 (or the entirety of the second cutting element 168) may comprise a second clade 172. As an even more specific example, the distal end of the first blade 170 may be sharpened and/or a distally facing end surface 170a of the first blade 170 (in whole or in part) may be arranged at a non-perpendicular angle with respect to the longitudinal axis A2 of the surgical device 140. Similarly, the distal end of the second blade 172 may be sharpened and/or a distally facing end surface 172a of the second blade 172 (in whole or in part) may be arranged at a non-perpendicular angle with respect to the longitudinal axis A2 of the surgical device 140. This angle (for the first blade 168 and/or the second blade 170) may be, for example: in a range between approximately (e.g., ±10% or ±5%) 15-75 degrees, or in a range between approximately (e.g., ±10% or ±5%) 30-75 degrees, or in a range between approximately (e.g., ±10% or ±5%) 30-60 degrees, or in a range between approximately (e.g., ±10% or ±5%) 40-50 degrees, or less than or equal to approximately (e.g., ±10% or ±5%) 75 degrees, or less than or equal to approximately (e.g., ±10% or ±5%) 60 degrees, or less than or equal to approximately (e.g., ±10% or ±5%) 50 degrees, or less than or equal to approximately (e.g., ±10% or ±5%) 45 degrees, or any other suitable angle. Furthermore, the angle of the first blade 170 may have mirror symmetry (or substantially mirror symmetry) with the angle of the second blade 172 across the longitudinal axis A2, as seen in FIGS. 8-10. As a result, the distally facing end surface 170a of the first blade 170 may be non-parallel to the distally facing end surface 172a of the second blade 172

The distally facing end surface 170a of the first blade 170 may be configured to create at least one continuous (e.g., uninterrupted) first cut through any one or combination of: the ex vivo portion 16 of the percutaneous implant 10, the in vivo portion 18 of the percutaneous implant 10, the tubular portion 28 of the percutaneous implant 10, and the anchor portion 30 of the percutaneous implant 10. Similarly, the distally facing end surface 172a of the second blade 172 may be configured to create at least one continuous (e.g., uninterrupted) second cut through any one or combination of: the ex vivo portion 16 of the percutaneous implant 10, the in vivo portion 18 of the percutaneous implant 10, the tubular portion 28 of the percutaneous implant 10, and the anchor portion 30 of the percutaneous implant 10. Furthermore, the first blade 170 and/or the second blade 172 may be constructed of any suitably rigid and/or hard material including, for example, metal (e.g., stainless steel and/or surgical steel), certain plastics (e.g., polycarbonate), ceramic, or any other suitable material, or any combination thereof.

A method of using the surgical device 140 to cut the percutaneous implant 10 is similar in many respects to the above-described method of using the surgical device 40 to cut the percutaneous implant 10. Differences include, for example, that as an initial (optional) step the guidewire 164 may be inserted through the opening 34 in the percutaneous implant 10 and into, for example, the stomach 14. If not already done so, the guidewire 164 may be inserted through the opening 162 in the surgical device 140 (starting at the distal end 144 of the surgical device 140). Next, while holding the guidewire 164 stationary or substantially stationary with respect to the patient, the surgical device 140 may be guided in the distal direction along the guidewire 140 toward the patient's body, such that the third arm 160 is inserted into the opening 34 of the percutaneous implant 10. Further distal movement of the surgical device 140 may cause the first blade 170 and the second blade 172 to create, respectively, a first cut and a second cut in the percutaneous implant 10. The first and second cuts may be parallel to each other and/or parallel to the longitudinal axis A1 of the percutaneous implant 10. Furthermore, the first and second cuts may be created simultaneously or substantially simultaneously in a single distal motion or pass of the surgical device 140.

FIGS. 11-16 depict a third embodiment of a surgical device, denoted with reference numeral 240. Various elements of the surgical device 240 illustrated in FIGS. 11-16 may be similar or identical in structure, configuration, and/or function to elements of the surgical device 40 described above in conjunction with FIGS. 2-7. Such elements are assigned with the same reference numeral as used in FIGS. 2-7, except incremented by 200. A description of some of these elements is abbreviated or eliminated in the interest of conciseness. Details of the structure, configuration, and/or function that differentiate the embodiments of the surgical device 240 illustrated in FIGS. 11-16 from the embodiment of the surgical device 40 in FIGS. 2-7 are the focus of the discussion below.

Material that has been cut away from the percutaneous implant 10 by the surgical device has the potential to build up behind or proximal to the cutting element as the cutting element moves forwardly or distally with respect to the percutaneous implant 10. This build-up of material has the potential to impede forward or distal movement of the surgical device, thereby requiring a user to exert additional force to cut the percutaneous implant 10. To mitigate this issue, the surgical device 240 may incorporate a first guide surface 260 and/or a second guide surface 262, as seen in FIGS. 11-13.

The first guide surface 260 is configured to guide at least a portion of the percutaneous implant 10 cut by the first cutting element 252 in a direction generally away from the longitudinal axis A2 of the surgical device 240 during use of the surgical device 240 to cut the percutaneous implant 10. For example, as the cutting element 252 moves distally along and cuts the tubular portion 28 of the percutaneous implant 10, the cut portion passing off of the cutting element 252 may come into contact with and then slide along the first guide surface 260. Due to, for example, its orientation and/or positioning, the first guide surface 260 may guide the cut portion of the percutaneous implant 10 away from the longitudinal axis A2 of the surgical device 240 as the cut portion of the percutaneous implant 10 slides along the first guide surface 260. As an example, the first guide surface 260 may guide the cut portion in a direction laterally to the side of the surgical device 240, at a non-perpendicular angle with respect to the longitudinal axis A2 of the surgical device 240.

The first guide surface 260 may be arranged proximal to at least a portion of the cutting element 252. As an example, the first guide surface 260 may be arranged proximal to at least the distally facing end surface 254a of the blade 254 of the cutting element 252. A distal end of the first guide surface 260 may be disposed adjacent to a first lateral side surface 264 of the cutting element 252. As a more specific example, the distal end of the first guide surface 260 may be immediately adjacent to or adjoin the first lateral side surface 264 of the cutting element 252 such that there is little or no gap between the first guide surface 260 and the first lateral side surface 264 of the cutting element 252. In alternative embodiments, the first guide surface 260 and the first lateral side surface 264 of the cutting element 252 may be integrally formed to define a single unitary surface. A proximal end of the first guide surface 260 may be disposed adjacent to and/or adjoin a first lateral side surface 268 of the body of the surgical device 240.

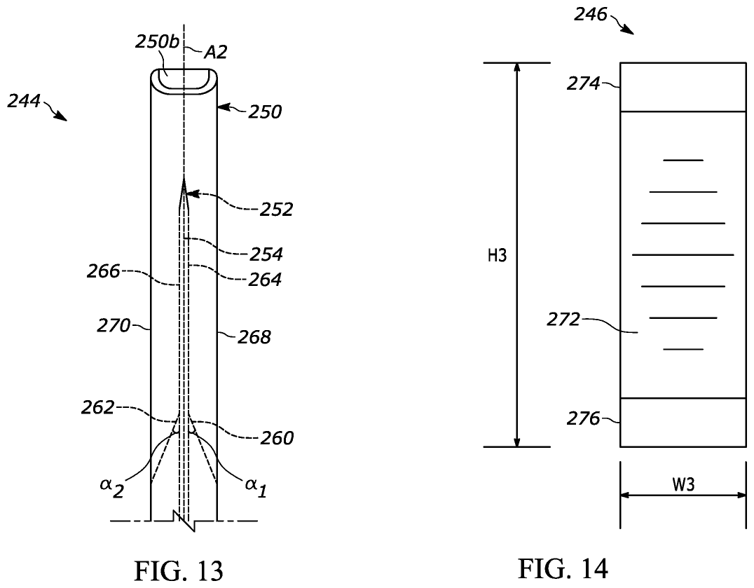

FIG. 13 shows a top view of the distal end 44 of the surgical instrument 240, with the cutting element 252, the first guide surface 260, and the second guide surface 262 shown in broken lines because they are obstructed from view by the second arm 250. As seen in FIG. 13, the first guide surface 260 may be partially or entirely arranged at an angle α₁ with respect to the longitudinal axis A2 of the surgical device 240. The angle α₁ may be less than 90 degrees such that the first guide surface 260 or at least a portion thereof is not perpendicular to the longitudinal axis A2. For example, the angle α₁ may be: less than or equal to approximately (e.g., ±10% or ±5%) 45 degrees, or less than or equal to approximately (e.g., ±10% or ±5%) 40 degrees, or less than or equal to approximately (e.g., ±10% or ±5%) 35 degrees, or less than or equal to approximately (e.g., ±10% or ±5%) 30 degrees, or less than or equal to approximately (e.g., ±10% or ±5%) 25 degrees, or less than or equal to approximately (e.g., ±10% or ±5%) 20 degrees, or any other suitable angle.

While the illustrated embodiment of the first guide surface 260 is substantially planar, in other embodiments, the first guide surface 260 may be curved or some combination of planar and curved, or any other shape suitable for guiding the cut portion of the percutaneous implant 10 away from the longitudinal axis A2 of the surgical device 240 during cutting.

The second guide surface 262 may have a similar configuration as the first guide surface 260, except mirrored across the longitudinal axis A2. Accordingly, the description above with respect to the first guide surface 262 applies to the second guide surface 262, within reason. The distal end of the second guide surface 262 may be disposed adjacent to a second lateral side surface 266 of the cutting element 252, and the second guide surface 262 may be arranged at an angle α₂ with respect to the longitudinal axis A2 of the surgical device 240. The first guide surface 260 may guide a first cut portion of percutaneous implant 10 in a first direction generally away from the longitudinal axis A2 of the surgical device 240, whereas the second guide surface 262 may guide a second cut portion of the percutaneous implant 10 in a second direction (different from the first direction) generally away from the longitudinal axis A2 of the surgical device 240.

The handle 246 at the proximal end 242 of the surgical device 240 may include features which may facilitate a user's grip during use of the surgical device 242. During cutting, a user may push the surgical device 240 in the distal direction at least partly with his or her thumb. To accommodate this, the handle 246 may include a concave surface 272 which generally faces in the proximal direction. In some embodiments, the concave surface 272 may have a curvature that approximates the curvature of the thumb of an average adult. The concave surface 272 may reduce the risk of the user's thumb sliding off of the proximal end 242 of the surgical device 240 while pushing the surgical device 240 in the distal direction during cutting, improve user comfort, and/or provide other benefits. Depending on the material used to construct the percutaneous implant 10, the user may need to overcome significant resistance to cut the percutaneous implant 10. The concave surface 272 may provide the user with greater confidence that they can exert the necessary force to cut the percutaneous implant 10 without losing grip of the surgical device 10.

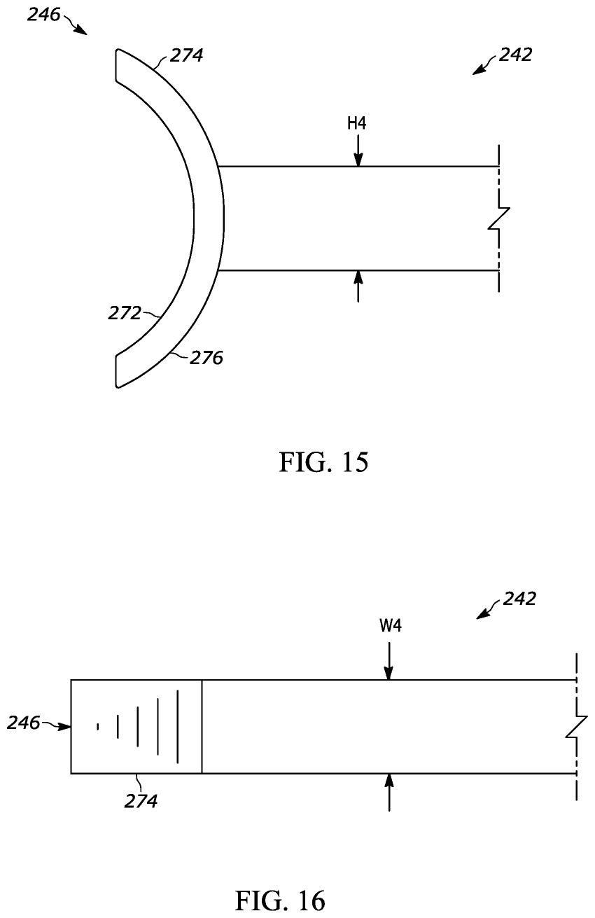

To further promote stability of the user's thumb, in some embodiments the handle 246 may include a width and/or height that exceeds that of a portion of the proximal end 242 of the surgical device 240 that is coupled to and/or adjoining the handle 246. As an example, an upper portion 274 of the handle 246 may extend or protrude above the portion of the proximal end 242 of the surgical device 240 that is coupled to and/or adjoining the handle 246, as seen in FIG. 14. Additionally or alternatively, a lower portion 276 of the handle 246 may extend or protrude below the portion of the proximal end 242 of the surgical device 240 that is coupled to and/or adjoining the handle 246. While the height H3 of the handle 246 may exceed a height H4 of the portion of the proximal end 242 of the surgical device 240 that is coupled to and/or adjoining the handle 246, a width W3 of the handle 246 may be the same as or substantially the same as the width W4 of the portion of the proximal end 242 of the surgical device 240 that is coupled to and/or adjoining then handle 246, as seen in FIGS. 15 and 16.

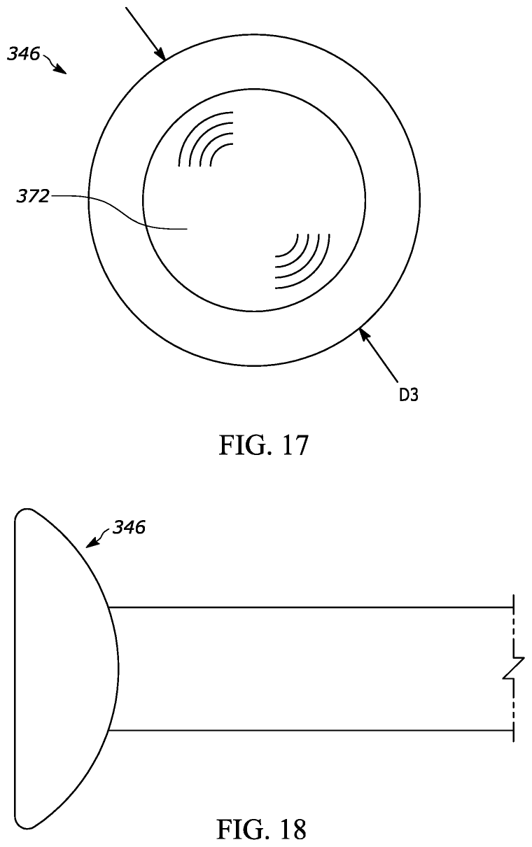

FIGS. 17 and 18 illustrate an alternative embodiment of the handle, identified with reference numeral 346, where both the width and height of the handle 346 exceed, respectively, the width and height of the portion of the proximal end 242 of the surgical device 240 that is coupled to and/or adjoining the handle 346. The handle 346 has a generally hemispherical shape, wherein the outer diameter D3 of this shape is greater than both the width and height of the proximal end 242 of the surgical device 240 that is coupled to or adjoining the handle 346. The inner surface of the generally hemispherical shape may define the concave surface 372 of the handle 346 and may be configured to receive at least the thumb of the user. The hemispherical shape may advantageously support the user's thumb on all sides, which may be useful depending on, for the example, the geometry of the object being cut by the surgical device 240.

While the foregoing describes the handles 246 and 346 primarily in the context of the surgical device 240, it should be understood that the handles 246 and 346 may be implemented in any of the embodiments of the surgical device described herein, including, for example, the surgical devices 40, 140, and 340.

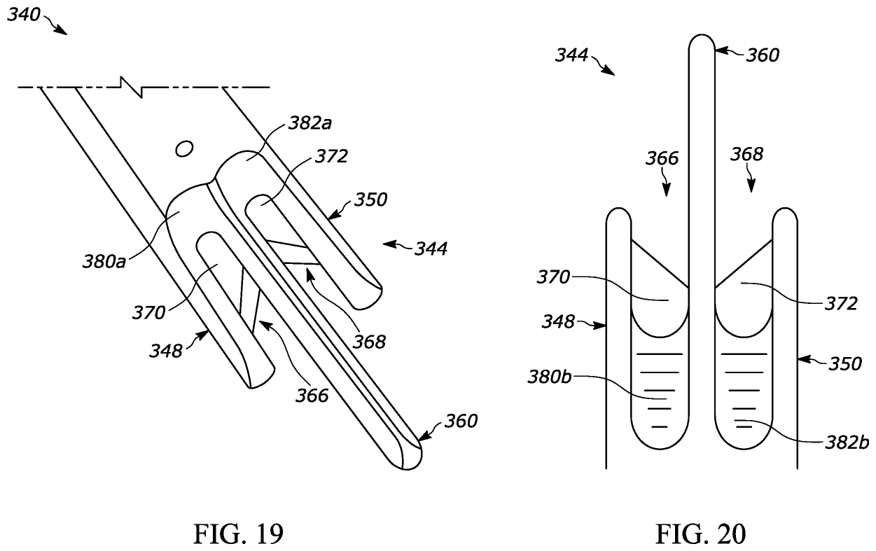

FIGS. 19-22 depict a fourth embodiment of a surgical device, denoted with reference numeral 340. Various elements of the surgical device 340 illustrated in FIGS. 19-22 may be similar or identical in structure, configuration, and/or function to elements of the surgical device 140 described above in conjunction with FIGS. 8-10. Such elements are assigned with the same reference numeral as used in FIGS. 8-10, except incremented by 200. A description of some of these elements is abbreviated or eliminated in the interest of conciseness. Details of the structure, configuration, and/or function that differentiate the embodiments of the surgical device 340 illustrated in FIGS. 19-22 from the embodiment of the surgical device 140 in FIGS. 8-10 are the focus of the discussion below.

Similar to the surgical device 240, the surgical device 340 may include guide surfaces for guiding material cut by the surgical device 340 generally away from the longitudinal axis A2 of the surgical device 340. As shown in FIG. 19, the surgical device 340 may include a first guide surface 380a arranged proximal to at least a portion of the first cutting element 352 and a second guide surface 382a arranged proximal to at least a portion of the second cutting element 368. The surgical device 340 may further include, as seen in FIG. 20, a third guide surface 380b and a fourth guide surface 382b which are mirror images of, respectively, the first guide surface 380a and the second guide surface 382a. The guide surfaces 380a-b and 382a-b are generally configured and/or function in a similar manner as the first guide surface 260 and/or second guide surface 262 of the surgical device 240. As such, the description above with respect to the first guide surface 260 and/or second guide surface 262 should be understood to apply, within reason, to the guide surfaces 380a-b.

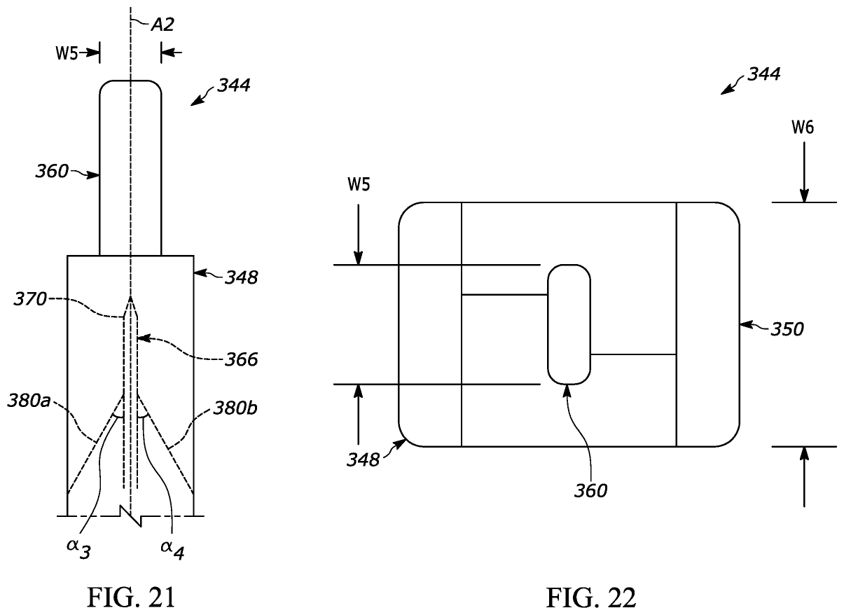

FIG. 21 shows a bottom view of the distal end 344 of the surgical instrument 340, with the first cutting element 366, the first guide surface 380a, and the third guide surface 380b shown in broken lines because they are obstructed from view by the first arm 348. As shown in FIG. 21, the first guide surface 380a may be partially or entirely arranged at an angle α₃ with respect to the longitudinal axis A2 of the surgical device 340, and the third guide surface 380b may be partially or entirely arranged at an angle α₄ with respect to the longitudinal axis A2 of the surgical device 340. The angle α₃ and/or angle α₄ may be less than 90 degrees such that the first guide surface 380a or at least a portion thereof is not perpendicular to the longitudinal axis A2 and/or such that the third guide surface 380b or at least a portion thereof is not perpendicular to the longitudinal axis A2. For example, the angle α₃ and/or the angle α₄ may be: less than or equal to approximately (e.g., ±10% or ±5%) 45 degrees, or less than or equal to approximately (e.g., ±10% or ±5%) 40 degrees, or less than or equal to approximately (e.g., ±10% or ±5%) 35 degrees, or less than or equal to approximately (e.g., ±10% or ±5%) 30 degrees, or less than or equal to approximately (e.g., ±10% or ±5%) 25 degrees, or less than or equal to approximately (e.g., ±10% or ±5%) 20 degrees, or any other suitable angle. It is noted that although the first guide surface 380a and the third guide surface 380b may not intersect with the longitudinal axis A2 of the surgical device 340, the first guide surface 380a and the third guide surface 380b are nevertheless angled with respect to the longitudinal axis A2 when viewed in two dimensions, for example, as seen in FIG. 21.

The second guide surface 382a may be orientated at a same or different angle as the first guide surface 380a. The fourth guide surface 382b may be oriented at a same or different angle as the third guide surface 380b.

Turning to FIG. 22, a width W5 of the third arm 360 or at least the distal end of the third arm 360 may be less than a width W6 of the first and second arms 348 and 350. This may facilitate insertion of the third arm 360 into the tubular portion 28 of the percutaneous implant 10 and/or lateral stability of the tubular portion 28 of the percutaneous implant 10 on the first arm 348 and/or second arm 350. In some embodiments, the width W5 of the third arm 360 may be less than or equal to an inner diameter of the tubular portion 28 of the percutaneous implant 10, whereas the width W6 of the first arm 348 and/or second arm 350 may be equal to or larger than an outer diameter of the tubular portion 28 of the percutaneous implant 10.

Any of the cutting elements described herein may be replaced, in alternative embodiments, with an ultrasonic cutting element, a rotating cutting element, or any other suitable cutting element including cutting elements having a blunt (rather than sharpened) cutting edge.

The foregoing embodiments of the surgical device 40, the surgical device 140, the surgical device 240, and the surgical device 340 are described as including two or more arms, which are parallel or substantially parallel to each other and/or the longitudinal axis A2. Alternative embodiments may arrange some or all of these arms, or portions thereof, such that they are non-parallel (e.g., angled) with respect to each other and/or the longitudinal axis A2 and/or include one or more chamfered surfaces to, for example, facilitate insertion of the surgical device 40, 140, 240, or 340 into the patient's body, facilitate insertion of the tubular portion 28 and/or another portion of the percutaneous implant 10 between the arms of the surgical device 40, 140, 240, or 340 and/or guide the percutaneous implant 10 toward one or more of the cutting elements of the surgical device 40, 140, 240, or 340. As an example, any one or any combination of the inner surface 48a of the first arm 48, an outer surface 48c of the first arm 48, the inner surface 50a of the second arm 50, and the outer surface 48c of the second arm 50 may be partially or entirely non-parallel or substantially non-parallel to the longitudinal axis A2. As a more specific example, moving in the distal direction, a portion or the entirety of the outer surface 48c of the first arm 48 may be angled inwardly toward the longitudinal axis A2 and/or a portion or the entirety of the outer surface 50c of the second arm 50 may be angled inwardly toward the longitudinal axis A2. This may, for example, facilitate insertion of the first arm 48 and/or second arm 50 into a stoma formed the abdominal wall 12 and/or stomach wall 26. Additionally or alternatively, moving in the distal direction, a portion or the entirety of the inner surface 48a of the first arm 48 may be angled outwardly away from the longitudinal axis A2 and/or a portion or the entirety of the inner surface 50a of the second arm 50 may be may be angled outwardly away from the longitudinal axis A2. This may, for example, facilitate insertion of a portion of or the entirety of the tubular portion 28 of the percutaneous implant 10 between the first arm 48 and the second arm 50, for example, guiding it toward the first cutting element 52. Similar angling and/or chamfering may also be applied to the inner surface 148a of the first arm 148, an outer surface 148c of the first arm 148, the inner surface 150a of the second arm 150, and the outer surface 148c of the second arm 150 of the surgical device 140. Additionally or alternatively, moving in the distal direction, the first surface 160a of the third arm 160 and/or the second surface 160b of the third arm 160 may be angled inwardly toward or outwardly away from the longitudinal axis A2.

Testing has demonstrated that the embodiments of the surgical device disclosed herein reduce the amount of force needed to remove a mushroom-retained gastrostomy tube. In one series of tests, a surgical device constructed in accordance with principles of the present disclosure was used to form a single axial cut in a mushroom-retained gastrostomy tube having an 20 Fr tube. The mean maximum force required to remove this tube after cutting was 9.5 lbs. In another series of tests, a surgical device constructed in accordance with principles of the present disclosure was used to form two coaxial cuts in a mushroom-retained gastrostomy tube having an 20 Fr tube. The mean maximum force required to remove this tube after cutting was 2.5 lbs. For comparison, another series of tests was performed where a mushroom-retained gastrostomy tube having an 20 Fr tube was removed without forming any cuts in the tube. Here, the mean maximum force required to remove the tube was 12.2 lbs. Thus, both the single-cut case and the two-coaxial-cut case were measured to have a lower maximum removal force than the un-cut case. Furthermore, the two coaxial cuts were found to facilitate folding of the mushroom retention cup and reduce the volume of material and amount of deformation required to fold through the simulated stoma tract. The mentioned tests involved an ex vivo model of an abdominal wall and a gastrostomy stoma fabricated with a low-friction plastic plate with a chamfered 11 mm hole through which the mushroom-retrained gastrostomy tube was introduced. The maximum force required for tube removal via traction was measured with a digital force sensor. The required forces for each sample group were compared using a paired t-test. High-frame rate video of the mushroom retention cup was recorded during removal to allow qualitative evaluation of the mechanisms of deformation.

### Exemplary Embodiments

Exemplary embodiments provided in accordance with the presently disclosed subject matter include, but are not limited to, the claims and the following embodiments:

Embodiment 1: A surgical device for cutting a percutaneous implant, the surgical device comprising: a proximal end and a distal end; wherein the proximal end comprises a handle; wherein the distal end comprises a first arm, a second arm, and a first cutting element disposed at least partially between the first arm and the second arm; and wherein at least a portion of at least one of the first arm and the second arm is distal to the first cutting element to prevent or inhibit the first cutting element from cutting tissue adjacent to the percutaneous implant during use of the surgical device to cut the percutaneous implant.

Embodiment 2: The surgical device of Embodiment 1, wherein the first cutting element comprises a first blade.

Embodiment 3: The surgical device of any one of Embodiments 1 to 2, wherein the first blade extends at least partially between an inner surface of the first arm and an inner surface of the second arm.

Embodiment 4: The surgical device of any one of Embodiments 1 to 3, wherein at least one of the first arm and the second arm is dimensioned to be inserted at least partially into an opening in the percutaneous implant.

Embodiment 5: The surgical device of Embodiment 4, wherein at least one of the first arm and the second arm has a width or diameter that is less than a width or diameter of the opening in the percutaneous implant.

Embodiment 6: The surgical device of any one of Embodiments 1 to 5, wherein at least a portion of the first arm is spaced from at least a portion of the second arm by a first distance, the first distance being larger than a wall thickness of at least a portion of the percutaneous implant to be cut by the surgical device.

Embodiment 7: The surgical device of any one of Embodiments 1 to 6, wherein a position of the first cutting element is fixed with respect to the first arm and the second arm during use of the surgical device to cut the percutaneous implant.

Embodiment 8: The surgical device of any one of Embodiments 1 to 7, wherein at least a portion of the first arm is parallel or substantially parallel to at least a portion of the second arm.

Embodiment 9: The surgical device of any one of Embodiments 1 to 8, further comprising a third arm having a proximal end and a distal end, the proximal end of the third arm being disposed between the first arm and the second arm, and the distal end of the third arm being distal to at least one of the first arm and the second arm.

Embodiment 10: The surgical device of Embodiment 9, wherein the third arm comprises an opening permitting a guidewire to pass through at least the third arm.

Embodiment 11: The surgical device of any one of Embodiments 9 to 10, wherein the at least a portion of the first cutting element is disposed between the first arm and the third arm.

Embodiment 12: The surgical device of any one of Embodiments 9 to 11, wherein the third arm is dimensioned to be inserted at least partially into an opening in the percutaneous implant.

Embodiment 13: The surgical device of any one of Embodiments 9 to 12, further comprising a second cutting element, wherein at least a portion of the first cutting element is disposed between the first arm and the third arm, and at least a portion of the second cutting element is disposed between the second arm and the third arm.

Embodiment 14: The surgical device of Embodiment 13, wherein a position of the first cutting element and a position of the second cutting element are fixed with respect to each other during use of the surgical device to cut the percutaneous implant.

Embodiment 15: The surgical device of any one of Embodiments 13 to 14, wherein the second cutting element comprises a second blade.

Embodiment 16: The surgical device of Embodiment 15, wherein the second blade extends at least partially between an inner surface of the second arm and a surface of the third arm.

Embodiment 17: The surgical device of any one of Embodiments 13 to 16, wherein a distally facing surface of the second cutting element is non-parallel to a distally facing surface of the first cutting element.

Embodiment 18: The surgical device of any one of Embodiments 1 to 17, further comprising a first guide surface proximal to at least a portion of the first cutting element and configured to guide at least a portion of the percutaneous implant cut by the first cutting element in a direction generally away from a longitudinal axis of the surgical device during use of the surgical device.

Embodiment 19: The surgical device of Embodiment 18, wherein at least a portion of the first guide surface is not perpendicular to the longitudinal axis of the surgical device.

Embodiment 20: The surgical device of Embodiment 19, wherein the at least a portion of the first guide surface is arranged at an angle of approximately 45 degrees or less with respect to the longitudinal axis of the surgical device.

Embodiment 21: The surgical device of any one of Embodiments 18 to 20, wherein a distal end of the first guide surface is adjacent to a lateral side surface of the first cutting element.

Embodiment 22: The surgical device of any one of Embodiments 9 to 17, further comprising: a first guide surface proximal to at least a portion of the first cutting element and configured to guide at least a first portion of the percutaneous implant cut by the first cutting element in a first direction generally away from a longitudinal axis of the surgical device during use of the surgical device; and a second guide surface proximal to at least a portion of the second cutting element and configured to guide at least a second portion of the percutaneous implant cut by the second cutting element in a second direction generally away from the longitudinal axis of the surgical device during use of the surgical device.

Embodiment 23: The surgical device of Embodiment 22, wherein at least a portion of the first guide surface and/or at least a portion of the second guide surface is not perpendicular to the longitudinal axis of the surgical device.

Embodiment 24: The surgical device of Embodiment 23, wherein the at least a portion of the first guide surface and/or the at least a portion of the second guide surface is arranged at an angle of approximately 45 degrees or less with respect to the longitudinal axis of the surgical device.

Embodiment 25: The surgical device of any one of Embodiments 22 to 24, wherein a distal end of the first guide surface is adjacent to a lateral side surface of the first cutting element, and wherein a distal end of the second guide surface is adjacent to a lateral side surface of the second cutting element.

Embodiment 26: The surgical device of any one of Embodiments 1 to 25, wherein the handle comprises a concave surface which faces generally in a proximal direction and is configured to receive at least a thumb of a user of the surgical device.

Embodiment 27: The surgical device of Embodiment 26, wherein the handle protrudes above and/or below a portion of the surgical device coupled to or adjoining the handle.

Embodiment 28: The surgical device of any one of Embodiments 26 to 27, wherein the handle comprises a substantially planar lateral side surfaces arranged on opposite sides of a longitudinal axis of the surgical device.

Embodiment 29: The surgical device of any one of Embodiments 26 to 27, wherein at least a portion of the handle has a generally hemispherical shape.

Embodiment 30: The surgical device of any one of Embodiments 1 to 29, wherein at least one of the first arm and the second arm comprises a surface that is non-parallel to a longitudinal axis of the surgical device and/or chamfered to facilitate insertion of the surgical device into a patient.

Embodiment 31: The surgical device of Embodiment 30, wherein the surface is configured to be inserted through a stoma in the patient without causing injury.

Embodiment 32: The surgical device of any one of Embodiments 1 to 31, wherein at least one of the first arm and the second arm comprises an opening permitting a guidewire to pass through the at least one of the first arm and the second arm.

Embodiment 33: The surgical device of any one of Embodiments 1 to 32, wherein the percutaneous implant comprises a percutaneous endoscopic gastrostomy (PEG) tube, a radiologically inserted gastrostomy (RIG) tube, and/or a surgically placed gastrostomy tube.

Embodiment 34: A method of cutting a percutaneous implant coupled to a patient, the method comprising: inserting at least a portion of a surgical device into an opening in an ex vivo portion of the percutaneous implant; and using the surgical device to create at least one continuous cut through the ex vivo portion and an in vivo portion of the percutaneous implant.

Embodiment 35: The method of Embodiment 34, wherein using the surgical device to create the at least one continuous cut comprises the surgical device cutting through the percutaneous implant without the surgical device cutting tissue adjacent to the percutaneous implant.

Embodiment 36: The method of any one of Embodiments 34 to 35, further comprising withdrawing the surgical device from the percutaneous implant and subsequently pulling the in vivo portion of the percutaneous implant out of the patient.

Embodiment 37: The method of any one of Embodiments 34 to 35, further comprising withdrawing the surgical device from the percutaneous implant and subsequently pushing the ex vivo portion of the percutaneous implant inside of the patient.

Embodiment 38: The method of any one of Embodiments 34 to 37, wherein using the surgical device to create the at least one continuous cut comprises moving the surgical device in a direction parallel or substantially parallel to a longitudinal axis of the percutaneous implant.

Embodiment 39: The method of any one of Embodiments 34 to 38, wherein the at least one continuous cut extends from a proximal end surface of the percutaneous implant to a distal end surface of the percutaneous implant.

Embodiment 40: The method of any one of Embodiments 34 to 39, wherein the surgical device comprises a proximal end and a distal end; wherein the proximal end comprises a handle; wherein the distal end comprises a first arm, a second arm, and a first cutting element disposed at least partially between the first arm and the second arm; and wherein at least a portion of at least one of the first arm and the second arm is distal to the first cutting element.

Embodiment 41: The method of Embodiment 40, further comprising inserting a guidewire through at least an opening in the first arm of the surgical device and the opening in the ex vivo portion of the percutaneous implant.

Embodiment 42: The method of Embodiment 40, wherein the at least a portion of the surgical device inserted at least partially into the opening in the ex vivo portion of the percutaneous implant further comprises a third arm disposed at least partially between the first arm and the second arm.

Embodiment 43: The method of Embodiment 42, wherein the surgical device further comprises a second cutting element, wherein at least a portion of the first cutting element is disposed between the first arm and the third arm, and at least a portion of the second cutting element is disposed between the second arm and the third arm.

Embodiment 44: The method of any one of Embodiments 43 to 43, further comprising, prior to inserting the third arm of the surgical device into the opening in the ex vivo portion of the percutaneous implant, inserting a guidewire through at least an opening in the third arm of the surgical device and the opening in the ex vivo portion of the percutaneous implant.

Embodiment 45: The method of any one of Embodiments 34 to 40, further comprising, prior to using the surgical device to create the at least one continuous cut through the ex vivo portion of the percutaneous implant, using the surgical device to deform at least a portion of the percutaneous implant

Embodiment 46: The method of any one of Embodiments 34 to 45, wherein the percutaneous implant comprises a percutaneous endoscopic gastrostomy (PEG) tube, a radiologically inserted gastrostomy (RIG) tube, and/or a surgically placed gastrostomy tube.

Embodiment 47: The method of any one of Embodiments 34 to 46, wherein the surgical device is the surgical device according to any one of Embodiments 1 to 46.

All features described herein, including in the specification, claims, abstract, and drawings, and all the steps in any method or process described herein, may be combined in any combination, except combinations where one or more of the features and/or steps are mutually exclusive.

While the present disclosure has been described in connection with various embodiments, it will be understood that the present disclosure is capable of further modifications. The present disclosure is intended to cover any variations, uses, or adaptations of the disclosed subject matter following, in general, the principles of the present disclosure, and including such departures from the present disclosure as, within the known and customary practice within the art to which the present disclosure pertains.

1. A surgical device for cutting a percutaneous implant, the surgical device comprising:
a proximal end and a distal end;
wherein the proximal end comprises a handle;
wherein the distal end comprises a first arm, a second arm, and a first cutting element disposed at least partially between the first arm and the second arm; and
wherein at least a portion of at least one of the first arm and the second arm is distal to the first cutting element to prevent or inhibit the first cutting element from cutting tissue adjacent to the percutaneous implant during use of the surgical device to cut the percutaneous implant.

2. The surgical device of claim 1, wherein the first cutting element comprises a first blade.

3. The surgical device of claim 1, wherein the first blade extends at least partially between an inner surface of the first arm and an inner surface of the second arm.

4. The surgical device of claim 1, wherein at least one of the first arm and the second arm is dimensioned to be inserted at least partially into an opening in the percutaneous implant.

5. The surgical device of claim 4, wherein at least one of the first arm and the second arm has a width or diameter that is less than a width or diameter of the opening in the percutaneous implant.

6. The surgical device of claim 1, wherein at least a portion of the first arm is spaced from at least a portion of the second arm by a first distance, the first distance being larger than a wall thickness of at least a portion of the percutaneous implant to be cut by the surgical device.

7. The surgical device of claim 1, wherein a position of the first cutting element is fixed with respect to the first arm and the second arm during use of the surgical device to cut the percutaneous implant.

8. The surgical device of claim 1, wherein at least a portion of the first arm is parallel or substantially parallel to at least a portion of the second arm.

9. The surgical device of claim 1, further comprising a third arm having a proximal end and a distal end, the proximal end of the third arm being disposed between the first arm and the second arm, and the distal end of the third arm being distal to at least one of the first arm and the second arm.

10. The surgical device of claim 9, wherein the third arm comprises an opening permitting a guidewire to pass through at least the third arm.

11. The surgical device of claim 9, wherein:
the at least a portion of the first cutting element is disposed between the first arm and the third arm;
the third arm is dimensioned to be inserted at least partially into an opening in the percutaneous implant; and/or
the surgical device comprises a second cutting element, wherein at least a portion of the first cutting element is disposed between the first arm and the third arm, and at least a portion of the second cutting element is disposed between the second arm and the third arm.

12. (canceled)

13. (canceled)

14. (canceled)

15. (canceled)

16. (canceled)

17. (canceled)

18. (canceled)

19. (canceled)

20. (canceled)

21. (canceled)

22. The surgical device of claim 9, further comprising:
a first guide surface proximal to at least a portion of the first cutting element and configured to guide at least a first portion of the percutaneous implant cut by the first cutting element in a first direction generally away from a longitudinal axis of the surgical device during use of the surgical device; and
a second guide surface proximal to at least a portion of the second cutting element and configured to guide at least a second portion of the percutaneous implant cut by the second cutting element in a second direction generally away from the longitudinal axis of the surgical device during use of the surgical device.

23. (canceled)

24. (canceled)

25. (canceled)

26. (canceled)

27. (canceled)

28. (canceled)

29. (canceled)

30. (canceled)

31. (canceled)

32. (canceled)

33. The surgical device of claim 1, wherein the percutaneous implant comprises a percutaneous endoscopic gastrostomy (PEG) tube, a radiologically inserted gastrostomy (RIG) tube, and/or a surgically placed gastrostomy tube.

34. A method of cutting a percutaneous implant coupled to a patient, the method comprising:
inserting at least a portion of a surgical device into an opening in an ex vivo portion of the percutaneous implant; and
using the surgical device to create at least one continuous cut through the ex vivo portion and an in vivo portion of the percutaneous implant.

35. The method of claim 34, wherein using the surgical device to create the at least one continuous cut comprises the surgical device cutting through the percutaneous implant without the surgical device cutting tissue adjacent to the percutaneous implant.

36. The method of claim 34, further comprising withdrawing the surgical device from the percutaneous implant and subsequently pulling the in vivo portion of the percutaneous implant out of the patient.

37. The method of claim 34, further comprising withdrawing the surgical device from the percutaneous implant and subsequently pushing the ex vivo portion of the percutaneous implant inside of the patient.

38. The method of claim 34, wherein using the surgical device to create the at least one continuous cut comprises moving the surgical device in a direction parallel or substantially parallel to a longitudinal axis of the percutaneous implant.

39. (canceled)

40. The method of claim 34,
wherein the surgical device comprises a proximal end and a distal end;
wherein the proximal end comprises a handle;
wherein the distal end comprises a first arm, a second arm, and a first cutting element disposed at least partially between the first arm and the second arm; and
wherein at least a portion of at least one of the first arm and the second arm is distal to the first cutting element.

41. (canceled)

42. (canceled)

43. (canceled)

44. (canceled)

45. (canceled)

46. The method of claim 34, wherein the percutaneous implant comprises a percutaneous endoscopic gastrostomy (PEG) tube, a radiologically inserted gastrostomy (RIG) tube, and/or a surgically placed gastrostomy tube.

47. (canceled)
